(19) United States
(12) Patent Application Publication
(10) Pub. No.: US 2006/0247781 A1
(43) Pub. Date: Nov. 2, 2006

(54) Implant

(75) Inventor: Tom J. Francis, Cordova, TN (US)

Correspondence Address: FITZPATRICK CELLA HARPER & SCINTO, 30 ROCKEFELLER PLAZA, NEW YORK, NY, 10112, US

(73) Assignee: SDGI HOLDINGS, INC., WILMINGTON, DE (US)

(21) Appl. No.: 11/117,485

(22) Filed: Apr. 29, 2005

(51) Int. Cl. A61F 2/44; A61F 2/02

(52) U.S. Cl. 623/17.16; 606/78

(57) ABSTRACT

An intervertebral disc nucleus replacement is provided that is configurable into a first configuration to be assumed while the replacement is implanted in the nuclear cavity and a second configuration to be assumed during the procedure of inserting the replacement into the nuclear cavity. The first configuration may have an accordion-like structure formed by a plurality of sections folded in an alternating, zigzag-like manner, and may be sized and shaped to conform to the nuclear cavity. The second configuration may be formed by straightening the plurality of folded sections into an unfolded, elongated, linear member, thereby affording a smaller effective cross-section. The nucleus replacement may be made of an elastic material having shape memory and may be formed so that the first configuration is an unmanipulated or relaxed configuration and the second configuration is a manipulated or unrelaxed configuration.

### BACKGROUND OF THE INVENTION

1. Field of the Invention

Embodiments of the present invention relate to an implant and, in particular, to an implant suitable at least for use as an intervertebral disc nucleus implant or replacement.

2. Description of the Related Art

In a normal human being or other vertebrate animal, intervertebral discs serve to dynamically stabilize the spine and to distribute forces between vertebral bodies. Referring to FIG. 1A, an intervertebral disc 10 includes a gelatinous nucleus pulposus 11, and an annulus fibrosis 12. The disc 10 is located between two vertebral end plates 16 (see FIGS. 4B and 4D). The nucleus pulposus 11 is laterally surrounded and confined by the annulus fibrosis 12. Both the nucleus pulposus 11 and the annulus fibrosis 12 are surrounded and confined above and below by the respective vertebral end plates 16. (For simplicity, the nucleus pulposus and the annulus fibrosis will hereinafter generally be referred to as the nucleus and the annulus, respectively.)

Intervertebral discs may be displaced or damaged due to, for example, trauma or disease. Disruption of the annulus 12, for example, a defect or tear, may allow the nucleus 11 to protrude (or “slip”) into the vertebral canal, as shown in FIG. 1B, a condition commonly referred to as a herniated, ruptured or slipped disc. The extruded nucleus 11 may affect a spinal nerve 13, which may result in nerve damage, pain, numbness, muscle weakness and/or paralysis. Intervertebral discs may also deteriorate due to the normal aging process. Over time, the disc dehydrates and hardens, reducing the height of the disc space. The reduction of the disc height, or spacing between adjacent vertebrae, leads to instability of the spine, decreased mobility and pain.

One way to relieve the symptoms of these conditions is to surgically remove a portion or all of the intervertebral disc. FIG. 1C shows a case in which the nucleus 11 has been removed, through a naturally occurring or artificially induced opening 14 in the annulus 12, leaving the nuclear cavity 15 empty. However, removal of the damaged or unhealthy disc may allow the disc space to collapse, which would lead to instability of the spine, abnormal joint mechanics and nerve damage, as well as severe pain. Therefore, after removal of the disc, the adjacent vertebrae surrounding the disc space were, in the prior art, typically fused together to preserve the disc space. Several devices exist to fill an intervertebral space following removal of all or part of the intervertebral disc in order to prevent collapse of the disc space and to promote fusion of the adjacent vertebrae. While a certain degree of success with these devices has been achieved, full motion is typically never regained after such vertebral fusions.

Such drawbacks of the vertebral fusion procedure have led to the development of disc replacements, or implants, as an alternative solution. Many of these implants are complicated, bulky devices made of a combination of metallic and elastomeric components. Thus, implantation of such devices requires an invasive surgical procedure, and even these implants typically do not bring back the desired degree of normal functioning, e.g., the full range of motion desired.

Accordingly, disc nucleus replacement technology is undergoing continual development. As one recent example of this development, disc nucleus replacements have been formed from materials such as hydrogels, in an attempt to simulate the gelatinous material of the natural disc nucleus. However, many such hydrogel replacements have been subject to damage during implantation and, once implanted, have been known to migrate within the nuclear cavity and/or to be expelled from the nuclear cavity through the annular opening by which they were inserted or through some other annular opening caused by a defect or the like. Even migration, let alone expulsion, may reduce the effectiveness of the nucleus replacement, since proper position may be a factor in the ability of the replacement to perform its intended functions, e.g., bearing loads, providing stabilization, and absorbing shock.

There are other inherent problems in disc nucleus replacement technology as a medical solution. For example, there is generally a trade-off between, on the one hand, achieving a nucleus replacement sufficiently large to simulate the natural nucleus and, on the other hand, providing a minimally invasive procedure of implanting the replacement so as to minimize the attendant disruption and destruction of annular tissue.

A need therefore exists for a nucleus implant or replacement that overcomes the inherent drawbacks and difficulties of this technology and that provides improved performance of the functions of the natural nucleus. The present invention addresses this need.

### SUMMARY OF THE INVENTION

Embodiments of the present invention relate to an implant, such as a nucleus implant or replacement that, on the one hand, may be configured to have a sufficiently small cross-section to be implanted by a minimally invasive procedure and, on the other hand, may be configured to have a sufficiently large effective size and appropriate shape to conform to the natural size and shape of the nuclear cavity (or other space) once implanted. In the case of nucleus implants, such conformity serves to maintain natural disc height, or spacing between vertebrae, which in turn subserves the basic functions of the natural disc nucleus, permitting motion while providing load-bearing capacity, dynamic stabilization, distribution of vertebral forces and shock absorption. By minimizing the invasiveness of the implantation procedure, the integrity of the annulus may be better maintained, thereby again promoting functional performance of the implant and reducing the likelihood and probable extent of further spinal degradation. The structural design and material characteristics of the implant enhance performance of the functions of the natural nucleus, provide superior durability, and serve to resist migration within, and expulsion from, the nuclear cavity. Finally, the implant may be implanted in the nuclear cavity with relative ease.

These and other advantages of the present invention will be apparent from the description herein.

One embodiment of the present invention relates to an intervertebral disc nucleus pulposus implant, comprising a member for insertion, through an opening in an intervertebral disc annulus fibrosis, into an intervertebral disc space. The member is configurable into a first, predetermined configuration in which the member is folded at at least three positions, and a second configuration in which the member is not folded at at least one of the three positions. The second configuration is for the insertion of the member, the member being configurable back into the first configuration after the insertion.

Another embodiment of the present invention relates to an intervertebral disc nucleus pulposus implant, comprising a member for insertion, through an opening in an intervertebral disc annulus fibrosis, into an intervertebral disc space. The member is configurable into a first, unmanipulated configuration in which the member is folded at at least a first position and the member is reverse folded at at least a second position, and a second configuration in which the member is not folded at at least one of the first and second positions. The second configuration is for the insertion of the member, the member being configurable back into the first configuration after the insertion.

A further embodiment of the present invention relates to an implant comprising a member for insertion through an opening. The member is configurable into a first, unmanipulated configuration having a length and a width, and a second configuration having a cross-section, taken perpendicular to a longitudinal axis of the member, the cross-section having a length and a width. The second configuration is for the insertion of the member, the member being configurable back into the first configuration after the insertion. When the member is in the first configuration, the member comprises at least three sections extending transverse to the length of the member in the first configuration. The ratio of the width of the cross-section of the member in the second configuration to the length of the member in the first configuration is less than or equal to approximately 0.25.

### BRIEF DESCRIPTION OF THE DRAWINGS

FIG. 1A is an axial, or top, cross-sectional view of a nucleus pulposus and an annulus fibrosis in an intervertebral disc space, in the case of a normal, healthy disc.

FIG. 1B is an axial, or top, cross-sectional view of a nucleus pulposus and an annulus fibrosis in an intervertebral disc space, in the case of a ruptured or herniated (“slipped”) disc.

FIG. 1C is an axial, or top, cross-sectional view of a nuclear cavity and an annulus fibrosis in an intervertebral disc space, from which the nucleus pulposus has been removed.

FIG. 2 is a perspective view of an intervertebral disc nucleus implant, or replacement, in a fully folded, unmanipulated, relaxed, implantation configuration.

FIG. 3A is a horizontal cross-sectional view of the intervertebral disc nucleus implant, or replacement, of FIG. 2.

FIG. 3B is a front elevational view of the intervertebral disc nucleus implant, or replacement, of FIG. 2, in its implantation orientation.

FIG. 3C is a side elevational view of the intervertebral disc nucleus implant, or replacement, of FIG. 2, in an orientation perpendicular to its implantation orientation.

FIGS. 4A, 4B, 4C and 4D are views of the intervertebral disc nucleus implant, or replacement, of FIG. 2, implanted in the intervertebral disc space, in its implantation orientation. FIG. 4A is a top, perspective view of an implant in a disc in the cervical region of the spine, FIG. 4B is a front, perspective view of the same (but in which the annulus is not shown). FIG. 4C is a top, perspective view of an implant in a disc in the lumbar region of the spine. FIG. 4D is a close-up of the view shown in FIG. 4B (but in which the annulus and the vertebral end plates are shown in cut-away cross-section).

FIG. 5 is a top, perspective view of the intervertebral disc nucleus implant, or replacement, of FIG. 2, in the process of being inserted into the intervertebral disc space, and simultaneously folding up into its implantation configuration.

FIG. 6A is a side or elevational view of the intervertebral disc nucleus implant, or replacement, of FIG. 2, in a fully unfolded, unrelaxed, insertion configuration.

FIG. 6B is a top view of the intervertebral disc nucleus implant, or replacement, shown in FIG. 6A.

FIG. 6C is a cross-sectional view of the intervertebral disc nucleus implant, or replacement, shown in FIG. 6A, taken along the line 6C-6C.

FIG. 6D is a cross-sectional view of the intervertebral disc nucleus implant, or replacement, shown in FIG. 6B, taken along the line 6D-6D.

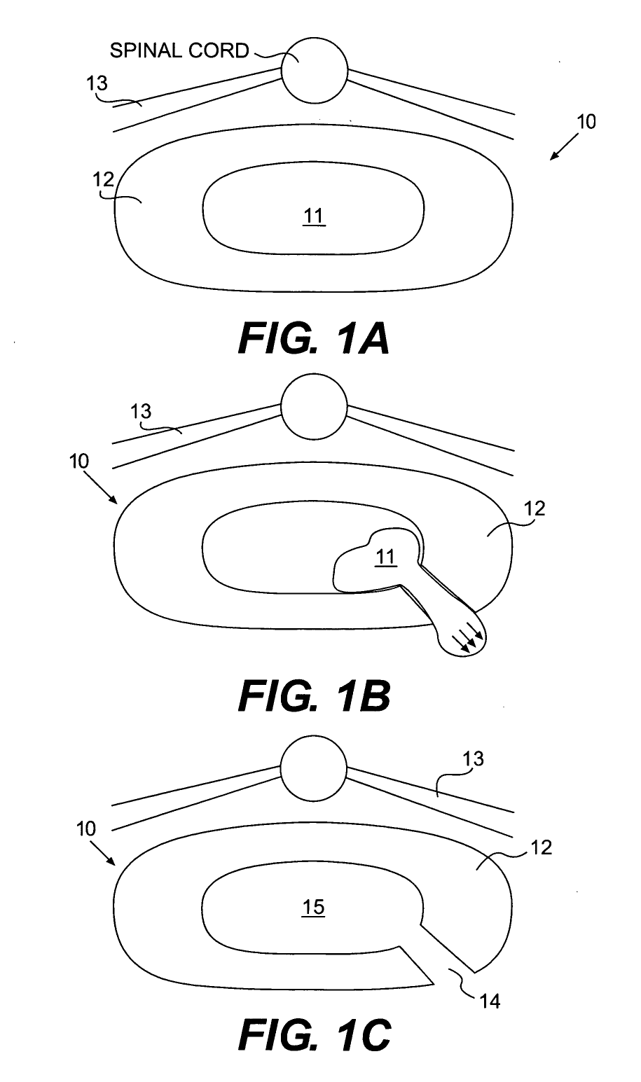

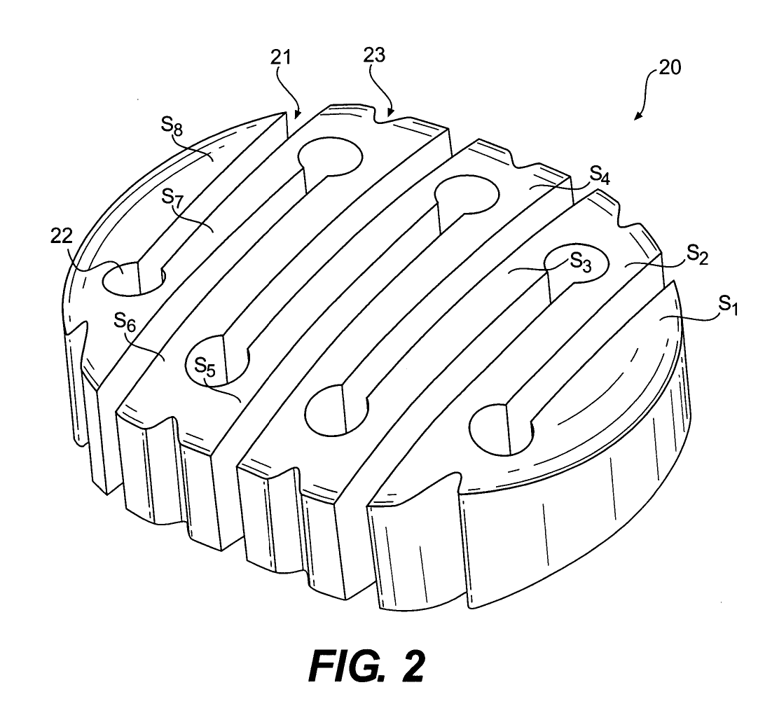

### DETAILED DESCRIPTION

While the present invention is described and illustrated in detail in the following description and accompanying drawings, the same is to be considered as illustrative and not restrictive in character, it being understood that only certain embodiments are discussed and shown herein. The invention is intended to encompass such modifications, equivalent arrangements, and applications of the principles of the invention as would be understood by those of ordinary skill in the pertinent arts to fall within the spirit and scope of the invention.

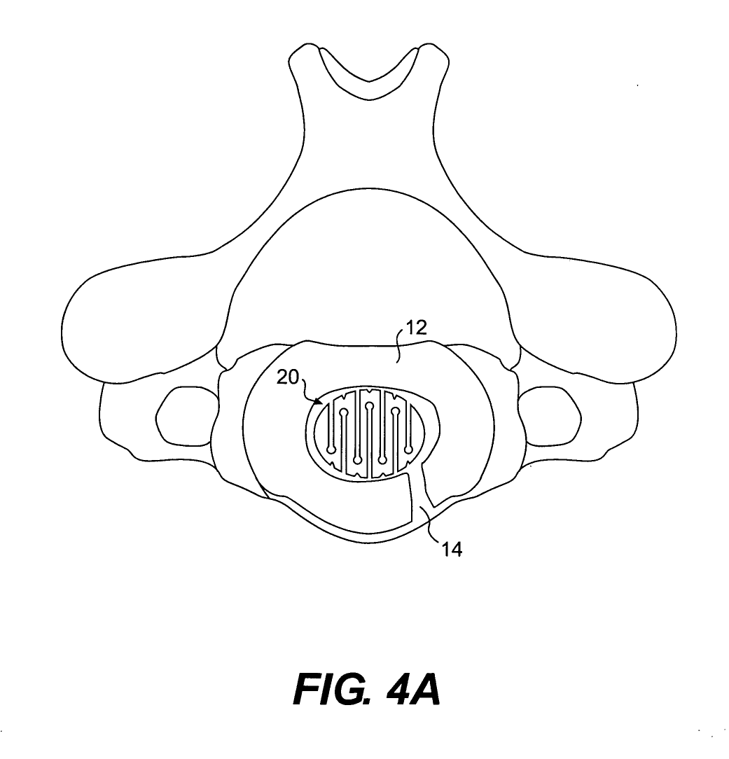

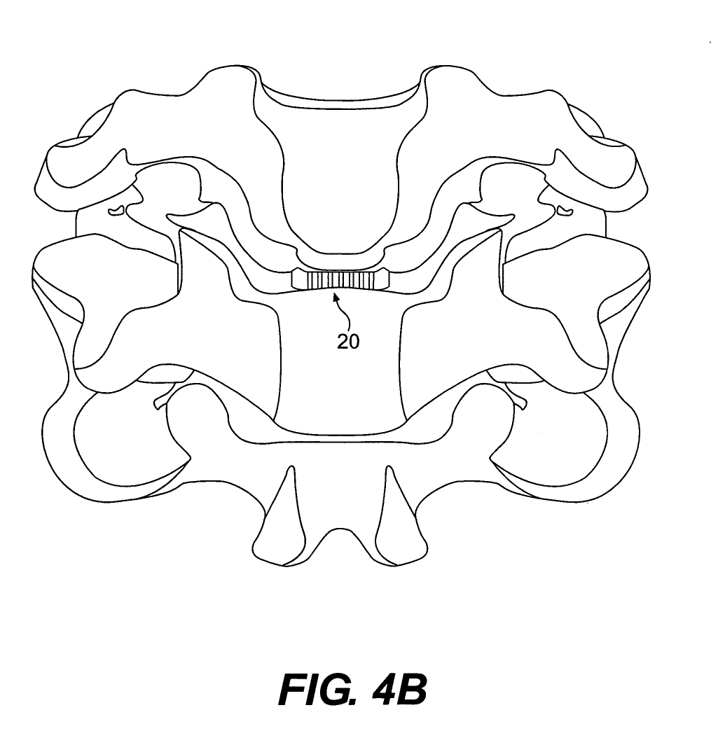

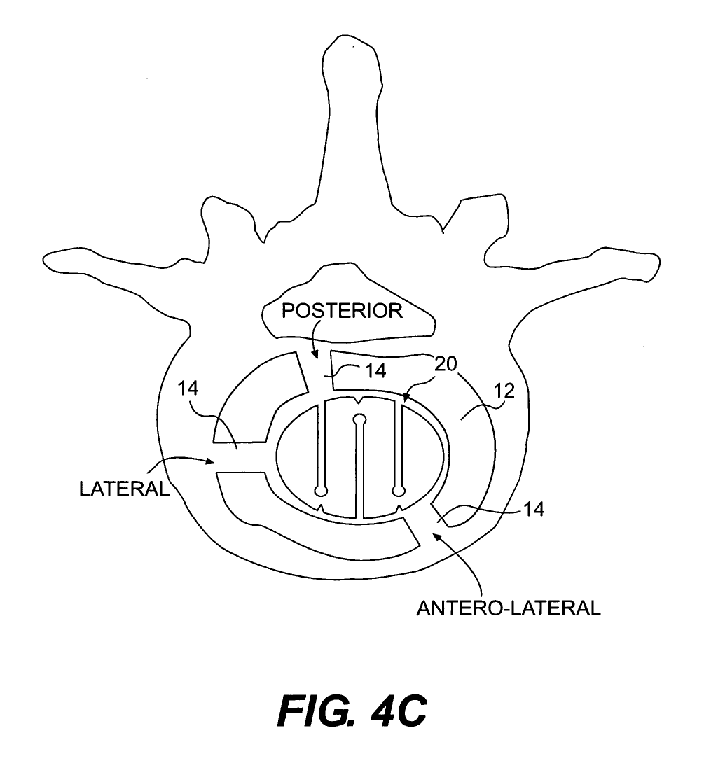

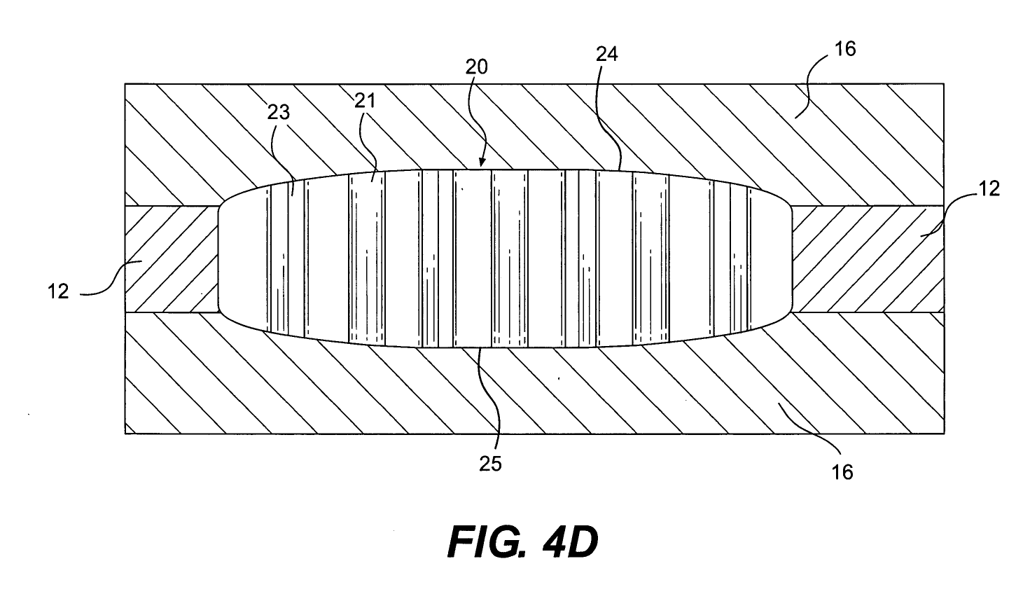

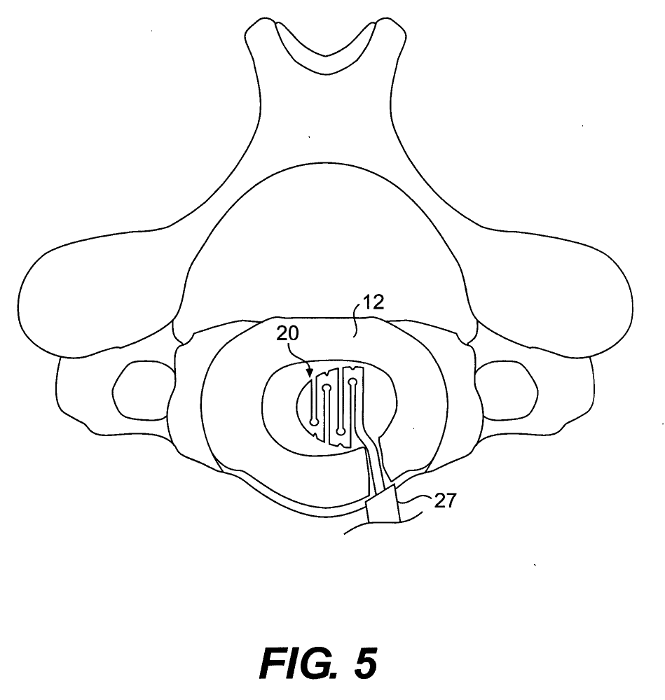

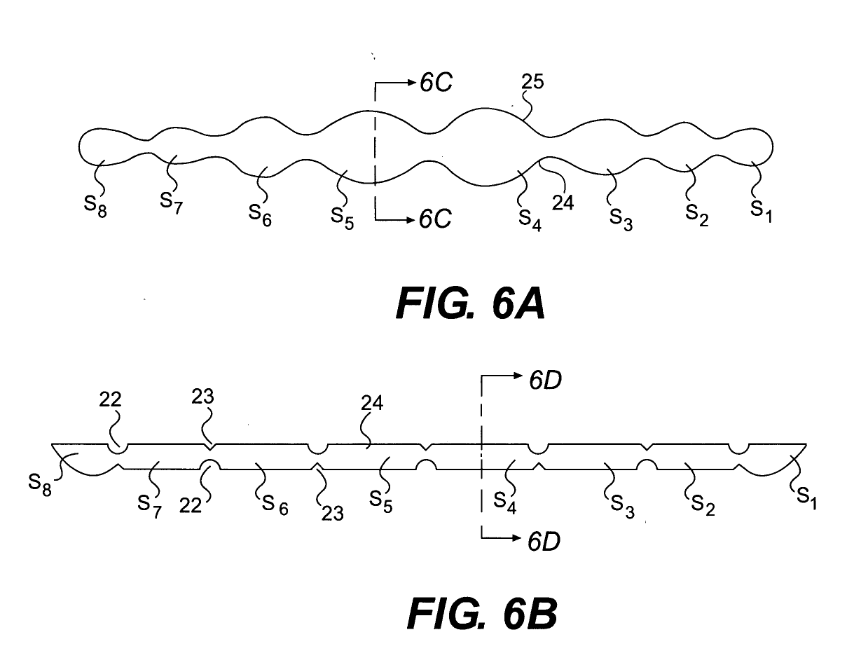

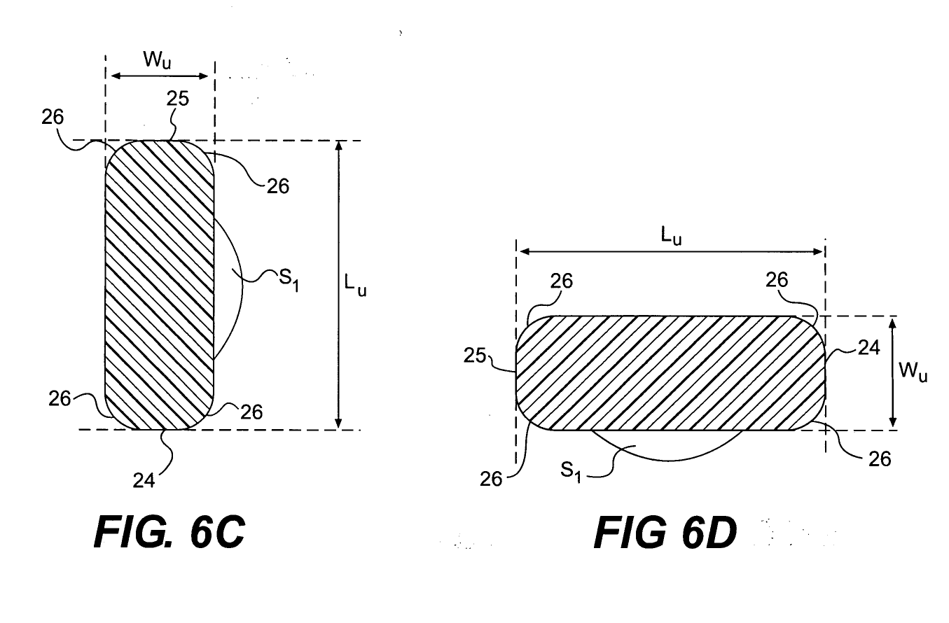

The structure and operation of a nucleus implant or replacement according to a first embodiment of the present invention will be explained with reference to the accompanying drawings. FIGS. 2, 3A-3C and 4A-4D show an implant 20 in a folded state, FIG. 5 shows the implant 20 in the process of being folded, and FIGS. 6A and 6B show the implant 20 in an unfolded state. Of these figures, FIGS. 4A-4D show the implant 20 in vivo, i.e., implanted in the nuclear cavity 15, and FIG. 5 shows the implant 20 in the process of being implanted, i.e., in the process of being inserted into the nuclear cavity 15. As illustrated in these figures, the implant 20 may be an elastic body capable of assuming different configurations, ranging from a fully folded state (shown in FIGS. 2, 3A-3C and 4A-4D) to a fully unfolded state (shown in FIGS. 6A and 6B).

The implant 20 may be formed in such a manner that the fully folded state is the (fully) relaxed state of the elastic body and the fully unfolded state is the (fully) unrelaxed state of the elastic body. (The terms “folded,” “unfolded,” “relaxed” and “unrelaxed” are used hereafter to refer to the fully folded, fully unfolded, fully relaxed, and fully unrelaxed configurations, respectively, unless otherwise indicated.) The elastic properties of the implant 20 may be such as to provide it with a “shape memory,” in the following sense. A force may be applied to the implant 20 to deform it from its relaxed state. When application of the force ceases, the implant 20 returns to its relaxed state, provided that the application of the force did not endure for an unduly long period of time. Thus, the relaxed state may be thought of as the unmanipulated state and the unrelaxed state as a manipulated state. Absent the application of external force, the implant 20 assumes its unmanipulated state. Under the application of external force, the implant 20 may be manipulated into a variety of manipulated states, in which the implant 20 is folded to different degrees. The implant 20 is manufactured in such a way that the unmanipulated state is the state that the implant 20 naturally or automatically assumes in the absence of (that is, prior or subsequent to) the application of an external force. In this sense, the unmanipulated state may also be deemed a predetermined state. (Of course, it is possible to apply multiple external forces that cancel each other out, whereby the implant 20 could remain in its unmanipulated configuration, even under the application of force.)

As shown in FIGS. 4A-4D, the unmanipulated, relaxed or folded configuration is the configuration that the implant 20 is designed to assume upon implantation and to retain for the duration of the period of implantation. The elongated, unfolded or unrelaxed, manipulated configuration is employed for the process of implanting, or inserting, the implant 20 into the nuclear cavity 15.

Thus, the implant 20 is not designed to have a single shape that is fixed once and for all, but rather can be manipulated or configured into different shapes and effective sizes. In this way, it is possible to provide the implant 20 with a first configuration that is optimized for purposes of in vivo operation and with a second, different configuration that is optimized for purposes of insertion. The implant 20 takes on the first configuration when it operates as an implant in vivo, and it takes on the second configuration when it is to be inserted into the nuclear cavity 15. Thus, for purposes of insertion the cross-section can be minimized (see FIGS. 6C and 6D), and for purposes of in vivo operation, the effective size and shape can be optimally adjusted to fit the nuclear cavity 15 (see FIGS. 4A and 4C). The implant 20 is thereby provided with a cross-section sufficiently small to be inserted into the nuclear cavity 15 by a minimally invasive procedure, but the implant 20 is also reconfigurable in such a manner that after insertion it folds up (becoming shorter but wider) so as to conform to the nuclear cavity 15. The multiple folds, or bends, of the implant 20 (shown in FIGS. 2, 3A, 4A and 4C) thus permit it to have a smaller cross-section for insertion than has heretofore been achieved in the prior art. In sum, since the implant 20 is manipulable or reconfigurable, both the insertion cross-section can be minimized and the in vivo shape and effective size can be optimized.

Thus, by virtue of this foldable, manipulable or reconfigurable design, whereby an elastic material having shape memory is employed and the implant 20 is formed to have the above-described relaxed and unrelaxed states, various advantages are provided. On the one hand, once implanted, the implant 20 in its folded configuration substantially fills and conforms to the nuclear cavity 15, which contributes to maintaining the natural disc height, or spacing between adjacent vertebrae. Disc height maintenance, in turn, subserves the biomechanical and other (e.g., physiological) functions of the disc (e.g., providing mobility and stability, bearing loads and absorbing shock). On the other hand, the unfolded or elongated configuration temporarily provides the implant 20 with a small cross-section, so that only a small opening 14 in the annulus 12 is required in order to insert the implant 20 (therethrough) into the nuclear cavity 15. By keeping the size of the required annular opening 14 small, the integrity of the annulus 12 is largely retained. This again assists in disc height maintenance and in normal disc functioning. Retention of annular integrity also serves to reduce the likelihood and probable extent of further spinal degradation, beyond the degree already provided by nucleus replacement.

In addition, since the overall cross-section of the implant 20 in its folded configuration may significantly exceed the area of the annular opening 14 through which it was inserted, the possibility of expulsion of the implant 20 from the nuclear cavity 15 may be greatly reduced. Again, since the implant 20 in its folded configuration may substantially fill and conform to the nuclear cavity 15, the possibility of unwanted migration within the nuclear cavity 15 may also be reduced.

It is intended that the implant 20 be implanted in the nuclear cavity 15 of the patient in the orientation shown in FIGS. 4A-4D, hereinafter referred to as the implantation orientation. If the implantation orientation is not achieved at the time of implantation, the implant 20 is designed to assume this orientation by virtue of the forces acting upon it due to the natural motion of the patient. In the following discussion, directional terms such as “top,” “bottom,” “front” and “rear,” will be used as referring to the implant 20 in the implantation orientation, regardless of which figure is being referred to, unless otherwise indicated.

As seen in FIGS. 2, 3A-3C, 4A-4D, in its folded state, the implant 20 may have an accordion-like shape, in which it is folded into a plurality of contiguous sections S₁, S₂, . . . Sₙ. (While the discussion herein generally treats of the case in which the implant 20 has eight sections S₁-S₈, other cases in which the implant 20 has other numbers of sections are also contemplated. FIG. 4C, discussed below, shows one such other example, in which the implant 20 has four sections S₁-S₄ (not labeled).) The folds alternate between ‘forward’ folds and ‘reverse’ folds so as to form the accordion-like, or zigzag-like, shape. That is, if sections S₁ and S₂ are connected at the front (foreground in FIG. 2) of the implant 20, then sections S₂ and S₃ are connected at the rear of the implant 20, sections S₃ and S₄ are connected at the front of the implant 20, and so on.

Between each two adjacent sections a gap in the form of a slot 21 is provided. Each slot 21 is open at one end, and closed at the other end. Adjacent slots 21 are open at opposite ends, so as to form an alternating pattern of open and closed slots 21 on either side of the implant 20, in correspondence to the alternating folds. The provision of these gaps improves the durability of the implant 20, by preventing contact between the sections. Without the slots 21, forces exerted upon the implant 20 resulting from natural bodily motion and loading would cause adjacent sections to rub or grind against one other, which could result in excessive wear.

At the closed end of each slot 21, a circular opening 22 is formed, such that each slot 21 resembles an elongated keyhole. At the connecting portion of any two adjacent sections, at the outside of the fold, across from circular opening 22, a substantially v-shaped cut-out or groove 23 is formed. The circular openings 22 and the grooves 23 facilitate the unfolding of the implant 20, relieving stress in the implant 20 when it is in the unfolded configuration and reducing the possibility of cracks, fractures, or other permanent deformations, which could otherwise occur when the implant 20 is unfolded and maintained in the unfolded configuration.

While the implant 20 is shown as having eight (or four) sections, it may be formed to have a different number of sections. While the sections S₁-Sₙ are shown as being parallel, they need not be. The number of folds, the angle of folding, and the size and shape of the gaps (slots 21) may be varied as appropriate. Assuming the size of the implant 20 in the folded configuration is kept constant, the number of folds or sections is inversely proportional to the size of the cross-section of the implant 20 when in its unfolded configuration (shown in FIGS. 6C and 6D). Accordingly, the implant 20 may be designed with a sufficient number of folds so as to permit the size of the cross-section of the unfolded implant 20 (e.g., the width W_{U} of the cross-section) to be kept to a desired minimum.

It is also understood that the circular openings 22 and the grooves 23, described above, represent only two specific examples of features that perform their functions. It will be appreciated by those of ordinary skill in the art that the circular openings 22 and the grooves 23 may be modified, for example, in shape and/or size, or replaced by other surface features or the like, such modified features and replacements being capable of performing the same functions. Some examples of such modifications and replacements may be found in U.S. Pat. No. 6,620,196 (directed to another nucleus implant), the entirety of which is hereby incorporated herein by reference.

Since the size of the nuclear cavity 15 varies across different regions of the spine, different vertebrate species, and different individuals, it is intended that the implant 20 may be manufactured in a variety of sizes, such that an appropriate one may be selected, based on its size in the folded configuration, so as to fit the particular nuclear cavity of the particular patient.

While the discussion so far has focused on the case in which the implant 20 is implanted in a nuclear cavity 15 of a disc 10 located in the cervical region of the spine (as shown in FIGS. 4A and 4B), it is contemplated that the implant 20 may be implanted in at least any of the cervical, thoracic and lumbar regions of the spine. FIG. 4C shows an example of the implant 20 implanted in a nuclear cavity 15 of a disc 10 located in the lumbar region of the spine. The size and cross-section of the implant 20 to be employed in any particular case may be varied in accordance with the anatomical variation along the length of the spinal column. For example, the discs increase in size from the cervical, through the thoracic, to the lumbar region, and it is expected that the size and cross-section of the implant 20 may increase correspondingly. (It is noted that FIG. 4C is not necessarily drawn precisely to scale with respect to FIG. 4A.) The implant 20 shown in FIG. 4C may have a larger effective size and cross-section in its folded configuration than the implant 20 shown in FIG. 4A.

Depending on where along the spinal column the implant 20 is to be implanted, the implant 20 may be implanted using a posterior, postero-lateral, antero-lateral, transforaminal, lateral, far lateral, anterior or any other clinically acceptable approach. Non-limiting exemplary approaches include an anterior approach for the cervical spine (shown in FIG. 4A), a posterior or transforaminal approach for the thoracic spine, and a posterior, lateral or antero-lateral approach for the lumbar spine (shown in FIG. 4C). (While FIG. 4C shows three possible approaches, it is understood that only one such approach would actually be employed to implant the implant 20.) The manner of implanting the implant 20 will be discussed further below.

The overall effective size and shape of the implant 20 in its folded configuration are designed so that the implant 20 in its folded configuration may substantially fill and conform to the size and shape of the nuclear cavity 15, and thus maintain contact with the annulus 12 and with the vertebral end plates 16. In this regard, the overall shape of the implant 20 in its folded configuration may be rounded on all sides.

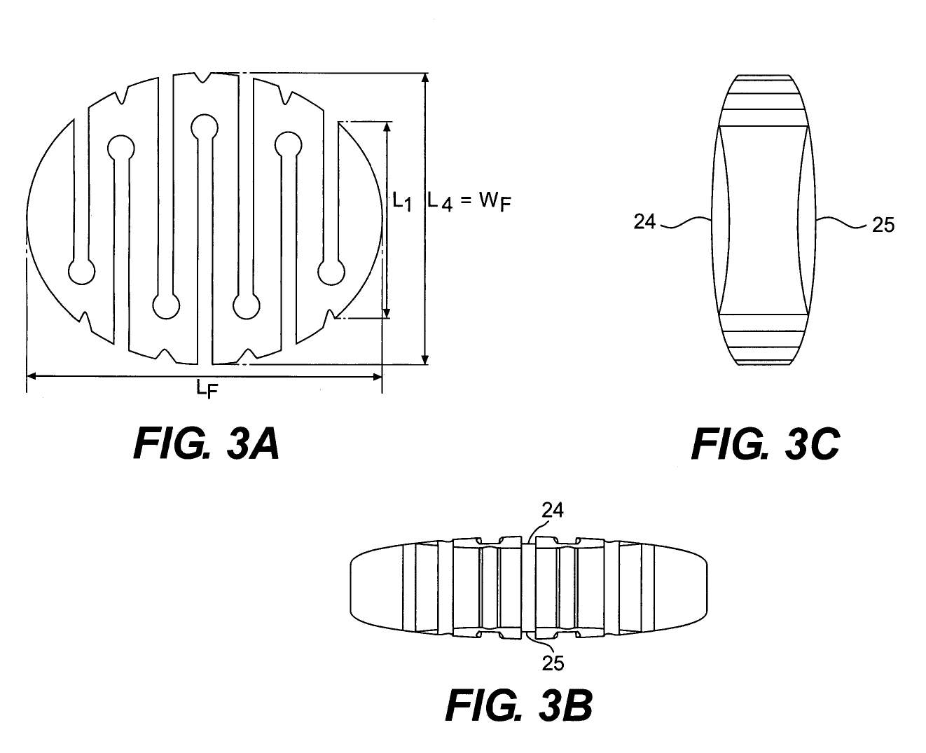

As seen, for example, in FIG. 3A, the horizontal cross-section of the implant 20 may have an elliptical shape, corresponding to the shape of the nuclear cavity 15 in the horizontal plane. Thus, the surfaces of the sections S₁-S₈ adjacent the grooves 23 (i.e., front and rear surfaces of the implant 20) are curved in the horizontal direction, following the elliptical shape of the horizontal cross-section. Approaching either the left or right end of the elliptical horizontal cross-section, the lengths of the sections S₁-S₈ gradually decrease. That is, the lengths of the centermost sections (e.g., L₄ and L₅) are the longest among the lengths, the lengths of the sections between the central region and the ends (e.g., L₂, L₃, L₆ and L₇) are shorter, and the lengths of the end sections (e.g., L₁ and L₈) are the shortest. (While L₂, L₃ and L₅-L₈ are not labeled in FIG. 3A, it will be understood that L₂ refers to the length of S₂, L₃ refers to the length of S₃, and so on.)

The equal lengths L₄ and L₅ may be considered the width W_{F} of the implant 20 in its folded configuration. The length of the implant 20 in its folded configuration is denoted by L_{F}. As a non-limiting example, the implant 20 can be formed so that, in its folded state, its width W_{F} may be approximately 8 mm and its length L_{F} may be approximately 10 mm. The width W_{F} may range from approximately 8 mm to approximately 22 mm, and the length L_{F} may range from approximately 10 mm to approximately 27 mm.

Viewed in perspective (FIG. 2), end sections S₁ and S₈ are seen to be truncated cylindrical portions, the cylindrical curvature coinciding with the elliptical shape of the horizontal cross-section, at the left and right sides of the implant 20. Thus, the vertical cross-section of end sections S₁ and S₈ have maximum areas at their longitudinal midpoints (i.e., the midpoints of L₁ and L₈) and minimum areas at their longitudinal endpoints (i.e., the endpoints of L₁ and L₈). In contrast, non-end sections S₂-S₇ have, along the greater part of their lengths, a vertical cross-section of constant area and shape. The maximum area of the vertical cross-section of either end section (S₁ or S₈) is greater than the area of the vertical cross-section of non-end sections S₂-S₇, and the minimum area of the vertical cross-section of either end section (S₁ or S₈) is smaller than the area of the vertical cross-section of non-end sections S₂-S₇.

In addition to the rounded, elliptical shape of the folded implant 20 in the horizontal plane, the folded implant 20 is also rounded in the vertical plane. As seen most easily from FIGS. 3B, 3C and 4D, the top surface 24 and the bottom surface 25 of the implant 20 may be formed to be convex, corresponding to the shape of the nuclear cavity 15 in the vertical plane. Accordingly, the top surface 24 and the bottom surface 25 of the implant 20 may conform to and contact the upper and lower vertebral end plates 16, respectively. Depending on the actual anatomy of the particular patient, such contact may not necessarily obtain over the entire top and bottom surfaces 24 and 25 of the implant 20. In addition, since the implant 20 is not fixed in place, other than by virtue of the conformity of its size and shape to the anatomical environment, it is expected that the degree of contact may vary slightly over time. (It is noted that the vertebral end plates 16 are covered with a cartilaginous film (not shown) such that, to speak more precisely, the implant 20 may be said to be in contact with this cartilaginous film rather than with the vertebral end plates 16. For simplicity, however, the implant 20 is described herein as contacting the vertebral end plates 16.)

Thus, the rounded shape of the implant 20, in both the horizontal and vertical dimensions, permits the implant 20 to substantially fill and conform to the nuclear cavity 15 and maintain contact with the annulus 12 and with the vertebral end plates 16. This helps the implant 20 perform the biomechanical and other functions of the natural disc nucleus more effectively. It also reduces the possibility of unwanted migration of the implant 20 within the nuclear cavity 15, which otherwise could impair the ability of the implant 20 to perform its functions.

FIGS. 6A and 6B show the nucleus replacement or implant 20 in its unfolded configuration. FIG. 6A shows a side or elevational view and FIG. 6B shows a top view. The implant 20 is placed in this configuration prior to being inserted into the nuclear cavity 15. As shown, in the unfolded configuration the implant 20 may have an elongated, substantially straightened, unbranched form. As shown in FIG. 6A, the implant in this configuration has a sinusoidal shape when viewed from the side. The amplitude of the sine wave is smallest at the ends of the unfolded implant 20 (i.e., toward end sections S₁ and S₈) and greatest at the central region of the unfolded implant 20 (i.e., at central sections S₄ and S₅). The increase in amplitude from the ends to the central region of the unfolded implant 20 is gradual, modest in magnitude, and symmetric from both ends to the central region. This sinusoidal shape of the implant 20 in its unfolded configuration provides the top and bottom surfaces 24 and 25, respectively, of the implant 20 with their respective convex shapes when the implant 20 is in its folded configuration, which feature was explained above with reference to FIGS. 3B, 3C and 4D.

Each of FIGS. 6C and 6D shows a cross-section of the implant 20 when it is in its unfolded configuration shown in FIGS. 6A and 6B, the cross-section being taken along the longitudinal axis of the unfolded implant 20, specifically, taken along the lines 6C-6C and 6D-6D, respectively. As shown, the cross-section may have a substantially rectangular shape, but with rounded short sides (corresponding to top and bottom surfaces 24 and 25) and rounded corners 26. Thus, the cross-section is a solid cross-section (i.e., it has no voids therein), and the perimeter of the cross-section is at least substantially smoothly continuous. As noted above, the maximum area of the cross-section of either end section (S₁ or S₈) is greater than the area of the cross-section of non-end sections S₂-S₇. Accordingly, when viewing the cross-sections shown in FIGS. 6C and 6D, the portion of the cross-section of end section S₁ that exceeds the cross-section of central section S₄ or S₅ is visible in the background as an arcuate section, shown here as non-cross-hatched region S₁.

The rounded short sides and the rounded corners 26 contribute, to some extent, to the convex shape the implant 20 has in its folded configuration, described above. In addition, the rounded short sides and the rounded corners 26, together with the unbranched form of the implant 20, facilitate smooth insertion of the implant 20 through the annular opening 14, minimizing the possibility of nicking, tearing or otherwise damaging the intact annular tissue. As stated above, the present invention provides the advantages of requiring a minimally sized annular opening 14, whereby the integrity of the annulus 12 may be largely maintained. As a non-limiting example, the implant 20 can be formed so that, in its unfolded state, its cross-section has a length L_{U} of approximately 5 mm and a width W_{U} of approximately 1.5 mm. The width W_{U} may range from approximately 1.5 mm to approximately 6 mm, and the length L_{U} may range from approximately 5 mm to approximately 8.5 mm.

As discussed above, one of the advantages of the invention is that for purposes of insertion the cross-section of the implant 20 can be minimized while for purposes of in vivo operation the effective size and shape of the implant 20 can be optimally adjusted to substantially fill and conform to the nuclear cavity 15. In that regard, based on the exemplary dimensions discussed herein, the ratio of the width W_{U} of the cross-section of the unfolded implant 20 to the length L_{F} of the folded implant 20 may be approximately 1.5 to 10 (=0.15), and the ratio of the width W_{U} of the cross-section of the unfolded implant 20 to the width W_{F} of the folded implant 20 may be approximately 1.5 to 8 (=0.1875). The ratio W_{U}/L_{F} may range from approximately 0.15 (e.g., 1.5 mm to 10 mm) to approximately 0.222 (e.g., 6 mm to 27 mm), and the ratio W_{U}/W_{F} may range from approximately 0.1875 (e.g., 1.5 mm to 8 mm) to approximately 0.273 (e.g., 6 mm to 22 mm).

The manner of implanting the nucleus replacement or implant 20 will be discussed with particular reference to FIG. 5. Prior to insertion of the implant 20, the disc is prepared for implantation (e.g., a portion or all of the natural nucleus 11 is removed, as appropriate, an opening 14 in the annulus 12 for insertion of the implant 20 is formed or enlarged, if necessary, etc.). The implant 20 is unfolded, for example, by grasping end sections S₁ and S₈ and pulling them apart from each other, either manually or by means of an appropriate instrument (not shown). Subsequent to or simultaneously with the unfolding of the implant 20, the implant 20 is loaded onto the same or another appropriate instrument 27 (shown schematically), which applies force to keep the implant 20 in its unfolded, unrelaxed configuration. Using the instrument 27, the implant 20 is inserted through the annular opening 14 into the nuclear cavity 15. As the implant 20 enters the nuclear cavity 15, it is released from the instrument 27, and consequently folds back up into its relaxed, folded state, as shown in FIG. 5. When the implant 20 folds up, sections S₁-S₈ fold in directions transverse to the longitudinal axis of the unfolded implant 20. When the implant 20 has fully exited the instrument, it will achieve its fully folded or relaxed configuration (shown in FIG. 4A and, in the case of an implant 20 having four sections S₁-S₄, in FIG. 4C). As stated, the implant 20 is designed to assume the implantation orientation upon insertion, upon subsequent adjustment by the surgeon performing the implantation, or upon natural motion of the patient.

As shown in FIGS. 4A-4D, in the implantation orientation, the longitudinal axes of the sections (S₁-S₈ or S₁-S₄) and of the slots 21 therebetween extend horizontally. Accordingly, the open ends of the slots 21 face the inner circumferential surface of the annulus 12, not the vertebral end plates 16 above and below the nuclear cavity 15. By virtue of this orientation of the accordion-like structure of the implant 20, the load-bearing capacity and stabilizing ability of the implant 20, among other functions, are promoted. For example, the possibility of elongation (unfolding) or collapse of the implant 20 under loading, which could in turn result in expulsion of the implant 20 from the nuclear cavity 15, is reduced by virtue of the accordion-like structure so oriented.

In order to determine whether the implant 20 is properly positioned in the nuclear cavity 15, or in the service of any other post-implantation examination, the implant 20 may be provided with any appropriate metallic components, e.g., beads, wire or the like, for x-ray identification thereof. As an example, tantalum beads (not shown) may be used as radiographic markers.

The nucleus replacement or implant 20 may be formed from any of a wide variety of biocompatible polymeric materials, including elastic materials, such as elastomeric materials, hydrogels or other hydrophilic polymers, or composites thereof. Other shape memory materials, such as shape memory alloys or shape memory polymers, may also be used. Examples and discussion of such materials may be found in U.S. Pat. No. 6,620,196, mentioned above, and it is understood that those of ordinary skill in the art would be apprised of the full range of materials that may be employed. A particular material that may advantageously be employed to form the nucleus replacement or implant 20 in this embodiment is PurSil™ (silicone polyether urethane), a thermoplastic elastomer.

In other embodiments of the invention, it is contemplated that the nucleus replacement or implant 20 may include any one or more of a number of features, as discussed below. More detailed explanations and examples of these features may generally be found in U.S. Pat. No. 6,620,196, mentioned above. It is understood that those of ordinary skill in the art will be apprised of the full range of variation these features may encompass.

The implant 20 may be provided with any of a variety of surface features, for example, physical patterns or chemical modifications (e.g., adhesive or other coatings). Such features may, for example, promote fixation of the implant 20, thereby enhancing resistance to migration and expulsion.

The implant 20 may be provided with an outer shell, sack or the like. In addition to helping to fix or anchor the implant 20, such a feature may serve to effectively seal annular openings or defects, to a greater degree than might be achieved by the implant 20 alone. This feature may also be employed to compensate for any differences in geometry and size between the implant 20 and the nuclear cavity 15, thereby improving fit. Such a shell or the like may also be resorbable, if desired, in which case it may be replaced in time by natural (e.g., scar) tissue, which may further anchor the implant 20 while preserving an appropriate degree of mobility for normal biomechanics.

The implant 20 may be provided with a supporting member, for example, to prevent excessive lateral (horizontal) deformation of the implant 20 such as might otherwise occur under conditions, for example, of high compressive loading. Such a supporting member will thus serve to maintain normal disc height. The supporting member will be strong but flexible, and may take the form of a jacket, band or the like. It may be substantially inelastic. It may also be made of a porous material to permit fluid circulation through the implant 20 in the case in which the implant 20 is composed of a material such as a hydrogel or other hydrophilic material.

The implant 20 may be provided with reinforcements, for example, in the area of the folds, to provide added strength so as to improve the structural integrity and further minimize the possibility of permanent deformation occurring due to the implant being unfolded.

The implant 20 may be provided with a locking feature, in the form, for example, of mating (complementary configured) sections, surface roughenings for friction fitting, or the like. Such a locking feature may further resist migration. In addition, such a feature could be employed to keep the implant 20 in the implantation configuration, for example, in cases in which the implant 20 is formed from a material that has little or no shape memory.

The implant 20 may be provided with the ability to deliver pharmacological agents. Pharmacological agents normally used in this context include growth factors (e.g., bone morphogenetic proteins) for repairing the annulus 12 and/or vertebral end plates 16, as well as drugs for treating various spinal conditions. Delivery of the pharmacological agents may be accomplished by any of a variety of means known in the art. For example, the agents may be dispersed within the implant 20, depending on the material composition of the implant 20, dispersed within a shell such as that discussed above, chemically attached to the surface of the implant 20, or otherwise associated with the implant 20. A porous material provided in the implant 20 or in associated components may be employed to release pharmacological agents.

In cases in which the implant 20 is formed of a material such as a hydrogel or other hydrophilic material, the implant 20 may be dehydrated to a desired degree prior to insertion, to be rehydrated after insertion, for example, by absorption of bodily fluids. Such dehydration can serve to minimize the size (e.g., cross-section) of the implant 20 for purposes of insertion. Accordingly, when employing dehydration, the number of folds provided to the implant 20 could be reduced.

Many different embodiments of the present invention may be constructed without departing from its spirit and scope. It should be understood that the present invention is not limited to the specific embodiments described and illustrated herein. To the contrary, the present invention is intended to cover all such modifications, applications and equivalent arrangements as fall within the spirit and scope of the invention as hereafter claimed. The scope of the claims is to be accorded the broadest interpretation so as to encompass all such modifications, and equivalent structures and functions.

What is claimed is:

1. An intervertebral disc nucleus pulposus implant, comprising:
a member for insertion, through an opening in an intervertebral disc annulus fibrosis, into an intervertebral disc space, said member being configurable into a first, predetermined configuration in which said member is folded at at least three positions, and a second configuration in which said member is not folded at at least one of the three positions, the second configuration being for the insertion of said member, said member being configurable back into the first configuration after the insertion.

2. An intervertebral disc nucleus pulposus implant according to claim 1,
wherein, in the first configuration, said member comprises a plurality of substantially elongate sections with a plurality of spaces provided therebetween, respectively, the plurality of substantially elongate sections including two end sections and at least one central section, each of the substantially elongate sections having a distal end and a proximal end, each central section being connected, at a proximal end thereof, to a proximal end of an adjacent section, and, at a distal end thereof, to a distal end of another adjacent section, whereby the sections are connected in a zigzag-like fashion, and
wherein, in the second configuration, said member is more linear than in the first configuration.

3. An intervertebral disc nucleus pulposus implant according to claim 1, wherein said member is formed of an elastic material that has shape memory, and wherein the first configuration is a relaxed configuration and the second configuration is an unrelaxed configuration.

4. An intervertebral disc nucleus pulposus implant, comprising:
a member for insertion, through an opening in an intervertebral disc annulus fibrosis, into an intervertebral disc space, said member being configurable into a first, unmanipulated configuration in which said member is folded at at least a first position and said member is reverse folded at at least a second position, and a second configuration in which said member is not folded at at least one of the first and second positions, the second configuration being for the insertion of said member, said member being configurable back into the first configuration after the insertion.

5. An intervertebral disc nucleus pulposus implant according to claim 4,
wherein, in the first configuration, said member comprises a plurality of substantially elongate sections with a plurality of spaces provided therebetween, respectively, the plurality of substantially elongate sections including two end sections and at least one central section, each of the substantially elongate sections having a distal end and a proximal end, each central section being connected, at a proximal end thereof, to a proximal end of an adjacent section, and, at a distal end thereof, to a distal end of another adjacent section, whereby the sections are connected in a zigzag-like fashion, and
wherein, in the second configuration, said member is more linear than in the first configuration.

6. An intervertebral disc nucleus pulposus implant according to claim 4, wherein, after insertion into the intervertebral disc space, said member is to assume the first configuration and to retain the first configuration while implanted in the intervertebral disc space.

7. An intervertebral disc nucleus pulposus implant according to claim 4, wherein said member is formed of an elastic material that has shape memory, and wherein the first configuration is a relaxed configuration and the second configuration is an unrelaxed configuration.

8. An intervertebral disc nucleus pulposus implant according to claim 4, wherein, when said member is fully unfolded, said member forms an unbranched, elongate member symmetric about a single longitudinal axis.

9. An intervertebral disc nucleus pulposus implant according to claim 4, wherein said member is for insertion into a space of the nucleus pulposus, so as to be able to be movably in contact with the annulus fibrosis and with two adjacent vertebral end plates, above and below the space of the nucleus pulposus, respectively.

10. An intervertebral disc nucleus pulposus implant according to claim 4, wherein, when said member is in the first configuration, said member has opposite convex surfaces, for conforming to a natural concavity of two adjacent vertebral end plates, which are disposed above and below said member when said member is implanted in the intervertebral disc space.

11. An intervertebral disc nucleus pulposus implant according to claim 4, wherein said member is capable of maintaining a natural spacing between two adjacent vertebrae.

12. An intervertebral disc nucleus pulposus implant according to claim 4, wherein, when said member is in the second configuration, said member has a cross-section, taken perpendicular to a longitudinal axis of said member, that has a width less than or equal to approximately 1.5 mm.

13. An intervertebral disc nucleus pulposus implant according to claim 4, wherein, when said member is in the second configuration, a cross-section of said member, taken perpendicular to a longitudinal axis of said member, has a substantially polygonal shape without voids therein, and has a perimeter that is at least substantially smoothly continuous.

14. An intervertebral disc nucleus pulposus implant according to claim 4, wherein the first configuration has a length and a width, and the second configuration has a cross-section, taken perpendicular to a longitudinal axis of said member, the cross-section having a length and a width, and
wherein the ratio of the width of the cross-section of said member in the second configuration to the length of said member in the first configuration is less than or equal to approximately 0.25.

15. An intervertebral disc nucleus pulposus implant according to claim 4, wherein said member is formed of a thermoplastic elastomer.

16. An implant, comprising:
a member for insertion through an opening, said member being configurable into a first, unmanipulated configuration having a length and a width, and a second configuration having a cross-section, taken perpendicular to a longitudinal axis of said member, the cross-section having a length and a width, the second configuration being for the insertion of said member, said member being configurable back into the first configuration after the insertion,
wherein, when said member is in the first configuration, said member comprises at least three sections extending transverse to the length of said member in the first configuration, and
wherein the ratio of the width of the cross-section of said member in the second configuration to the length of said member in the first configuration is less than or equal to approximately 0.25.

17. An implant according to claim 16,
wherein said at least three sections are substantially elongate sections with a plurality of spaces provided therebetween, respectively, the substantially elongate sections including two end sections and at least one central section, each of the substantially elongate sections having a distal end and a proximal end, each central section being connected, at a proximal end thereof, to a proximal end of an adjacent section, and, at a distal end thereof, to a distal end of another adjacent section, whereby the sections are connected in a zigzag-like fashion, and
wherein, in the second configuration, said member is more linear than in the first configuration.

18. An implant according to claim 16, wherein said member is formed of an elastic material that has shape memory, and wherein the first configuration is a relaxed configuration and the second configuration is an unrelaxed configuration.

19. An implant according to claim 16, wherein, when said member is fully unfolded, said member forms an unbranched, elongate member symmetric about a single longitudinal axis.

20. An implant according to claim 16, wherein, when said member is in the second configuration, the width of the cross-section is less than or equal to approximately 1.5 mm.

21. An implant according to claim 16, wherein the ratio of the width of the cross-section of said member in the second configuration to the length of said member in the first configuration is less than or equal to approximately 0.15.

22. An implant according to claim 16, wherein the implant is an intervertebral disc nucleus pulposus implant, for insertion, through an opening in an intervertebral disc annulus fibrosis, into an intervertebral disc space.
